(19) United States
(12) Patent Application Publication
(10) Pub. No.: US 2009/0099099 A1
(43) Pub. Date: Apr. 16, 2009

(54) Isoflavone Glycosides as Peroxisome Proliferator-Activated Receptor-alpha Modulator

(76) Inventors: Hong Wang, Somerville, MA (US); David Y-W Lee, Cambridge, MA (US)

Correspondence Address: Hong Wang, 18 Thorpe St., Apt. 2, Somerville, MA, 02143, US

(21) Appl. No.: 11/872,771

(22) Filed: Oct. 16, 2007

(51) Int. Cl. A61K 31/7048; C07H 3/02; A61P 3/04

(52) U.S. Cl. 514/23; 536/1.11

(57) ABSTRACT

A method of treating or preventing diseases related to modulation of PPAR-α in comprising administering to a human or other mammals in need of such treatment an effective amount of plant material derived from plants of the genera Puerila Lobata.

### FIELD OF INVENTION

We discovered that puerarnn [(4H-1-Benzopyran-4-one, 8-β-D-glucopyranosyl-7-hydroxy-3-(4-hydroxyphenyl), C₂₁H₂₀O₉], an isoflavone-C-glycoside isolated from Puerira Lobata, upregulates the nuclear transcription factor, peroxisome proliferator-activated receptor (PPAR)-α in human liver cells (HepG2), We are filing an application to patent the use of isoflavone glycosides as PPAR-α modulator.

### CROSS-REFERENCE TO RELATED APPLICATIONS

N/A

### STATEMENT REGARDING FEDERALLY SPONSORED RESEARCH OR DEVELOPMENT

N/A

### BACKGROUND OF THE lNVENTlON

### Peroxisoome Proliferator-Activated Receptors

Peroxisome proliferator-activated receptors (PPARs) are a group of nuclear receptor that play critical role in lipid metabolism, There are three isoforms (PPAR-α, β, and γ) encoded by separate genes, All PPARs are ligand-activated transcription factors that regulate gene expression through binding specific DNA sequences called peroxisome proliferator response elements (PPREs). in the presence of agonist, PPAR heterodimerizes with retinoid X receptor and binds to PPREs and recruits co-activators to activate target genes (Ferre, 2004).

PPAR-α is first discovered by lssemann and Green in 1990 (Issemann and Green, 1990). It is mainly expressed in tissues with high capacity for fatty acid oxidation such as liver, heart, kidney, intestine and muscle, In the liver, activation of PPAR-α induces expressions of many genes involves in fatty acid uptake, transportation and oxidation (Mandard et al., 2004). When activated, PPAR-α promotes fatty acid oxidation, ketone bodies synthesis and glucose sparing. Endogenous PPAR-α agonists include long chain fatty acids and eicosanoids such as hydroxyeicosatetraenoic acids, prostaglandins and leukotrienes (Mandard et al., 2004). Synthetic PPAR-α agonists include clofibrate, carbaprostacyclin, non-steroidal anti-inflammatory drugs, pirinixic acid, phthalate ester plasticizers and second generation fibrates (such as fenofibrate, bezafibrate and gemfibrozil) (van Raalte et al., 2004).

### Current Clinical Uses of PPAR-α Agonists

Synthetic ligands include fibrates, which are the drugs used in the treatment of hyperlipidemia (Kersten, 2002), The role of PPAR-α is to sense and respond to cellular lipid concentrations by regulating transcription of factors involved in the catabolism of those lipids (Morris et al., 2004). Clinically, fibrates such as clofibrates, fenofibrate, bezafibrate and gemfibrozil, have been used to treat hyperlipidemia and coronary heart disease. There is evidence that gemfibrozil therapy could benefit people with metabolic syndrome in lowering the risk of cardiac events (van Raalte et al., 2004).

### Other Potential Novel Uses of PPAR-α Agonists PPAR-α and Obesity

To further investigate the importance of PPAR-α transgenic mice deficient in PPAR-α was created. These mice have an impaired expression and inducton of several hepatic target genes and later developed obesity (Costet et al., 1998). PPAR-α deficiency leads to an original form of monogenic, late onset spontaneous obesity with stable caloric intake (Costet et al. 1998). PPAR-α deficient mice are not hyperphagic and the obesity pattern is a closer reminiscence of the pattern observed in human (Costet et al., 1998). Treatment with a synthetic PPAR-α agonist, fenofibrate, reduced adiposity in rats or mice fed with a high fat diet (Guerre-Millo et al., 2000; Mancini at al., 2001; Yoon et al., 2002; Yoon et al., 2003; Jeong et al., 2004; Ji et al., 2005). Overall, these results suggest that activating PPAR-α. thus increase the genes for enhancement of mitochondrial fatty acid oxidation in liver, may be an effective phenotype-based treatment strategy for dietary obesity (Ji et al., 2005). They also further illustrate the central role of PPAR-α in the development of obesity.

### Obesity

Obesity is defined as an excess of body fat. A surrogate marker for body content is the BMI, which is determined by weight (kilograms) divided by height squared (square meters). In clinical terms, a BMI of 25-29 kg/m² is called overweight; higher BMIs (≧30 kg/m²) are called obesity. The best way to estimate obesity in clinical practice is to measure waist circumference. This is because an excess of abdominal fat is most tightly associated with the metabolic risk factors.

The prevalence of overweight and obesity has increased markedly in the last two decades in the United States. Overall, among adults over age 20, 65.1% were overweight or obese (Hedley et al., 2004). There is no indication that the prevalence of obesity is decreasing despite the multitude of efforts by federal agencies trying to curtail the obesity epidemic. The high prevalence of obesity remains a major public concern and is accompanied by a large economic burden. It has been estimated that obesity accounts for $100 billion dollars in health care expenses per year and is responsible for 5.7% of US health care (Wolf and Colditz, 1998). Obesity is associated with several risk factors and diseases. These diseases include metabolic syndrome, diabetes, cardiovascular diseases and certain kinds of cancer,

Obesity is a chronic disease and involves a complex interplay between environment and genetic interaction. However, although genes play a certain role in obesity, epidemiological studies suggested that environmental factors play a significant role in the current prevalence of obesity since our gene pools in the US have not changed dramatically. Current treatment options include: dietary intervention, physical activity, behavior modification, pharmacotherapy and surgery (AGA, 2002). Although the most effective treatment among these options is surgery, it is reserved for those severely obese patients. All other single treatment is rarely successful. Caloric restriction, physical exercise and behavior modification constitute the standard model for obesity treatment. However, in most cases, these methods, either alone or in combination. are generally met with poor longAerm outcomes (NTFPTB, 1996). Therefore, pharmacological agents are an adjunct therapy to the treatment of obesity. Recent ant-obesity agents have had only limited success and some, such as fenfluramine and dextenfluramine, are unsatisfactory due to side effects and have been withdrawn from the market. Many dietary supplements currently on the market have no conclusive evidence that they are effective. They are also not well-characterized and their safety is a major concern (Dwyer et at., 2005). Therefore, an agent with anti-obesity activity and few side effects) such as PPAR-α agonists or modulators) would be important agents for the treatment of obesity.

### PPAR-α and Inflammation

Besides the control of lipid metabolism, activation of PPARs could modulate many inflammatory response (Devchand et al., 1996). PPAR-α displays anti-inflammatory activities and controls the inflammatory response in vascular wall and liver. PPAR-α activation impairs inflammatory cytokine signaling pathways and can suppress the activities of important transcription factor, NF-κb, which regulates genes implicated in inflammation (Delerive et at., 2000 Zambon et al.) 2006). Interestingly, as mentioned above, non-steroidal anti-inflammatory drugs (NSAIDs) including aspirin and ibuprofen can activate PPAR-α. Part of the anti-inflammatory actions of NSAIDs may come from activation of PPAR-α. Therefore, PPAR-α agonists or modulators could be use as anti-inflammatory agents. Recent studies have showed that the anti-inflammatory actions of PPAR-α agonist could be used as a neuroprotective agents for the treatment of stroke and possibly other neurodegenerative diseases including Alzheimer's disease, Parkinson's disease and multiple sclerosis (Deplanque et al., 2003; Bordet et al., 2006).

### Natural Isoflavone Glycoside, Puerarin

Puerarin [(4H-1-Benzopyran-4-one, 8-β-D-glucopyranosyl-7-hydroxy-3-(4-hydroxyphenyl), C₂₁H₂₀O₉], an isoflavone glycoside is isolated from Puerira Lobata (Radix puerariae, or Kudzu) (FIG. 1). Puerira Lobata is one of the ancient Chinese medicinal plants used for diabetic disease. It is a perennial leguminous vine of the genus and has been used also as food in Japan and China. The root of kudzu (Radix puerariae RP) was first described in the Chinese material medica (Sheng Nong Ben Cao Jing, 1278AD) as sweet and acrid in taste, cool in nature and was used as an antipyretic, antidiarrhetic, diaphoretic and anti-emetic agent (Keung and Vallee, 1998). Kudzu was also listed in the most comprehensive medical book of the time named Grand Materia Medica-(Ben Cao Gang Mu), compiled by Li Shi Zhen (Li. 1596), in which kudzu was described for the treatment of diabetes mellitus (DM).

RP was recommended for use to treat stiffness and pain of the neck, febrile disease and for the induction of the measles eruption in the texts of traditional Chinese is medicine (Keung and Vallee, 1998). Puerarin, the major ingredient of RP, has been clinically tested in cardiovascular diseases based on its pharmacological action, which includes: increased cerebral and cardiovascular circulation (Qicheng, 1980). Puerarin has many beneficial effects for patients with heart failure, myocardial infarction and angina (Duan et al., 2000; Luo and Zheng, 2001; Xiao et al., 2004). The following table summarized the clinical uses of puerarin in cardiovascular diseases in humans.

TABLE 1

| Subjects | Effects | Reference |
|---|---|---|
| Chronic heart failure | ↓ oxidized-LDL, improve heart | (Duan et al., |
| patients | function | 2000) |
| Acute myocardial infarction | Improve rennin, angiotensin and | (Li et al., 1997) |
| (AMI) patients, | endothelin profile. | |
| Unstable angina (UA) | ↓ platelet granule membrane protein | (Luo and Zheng, |
| patients, | (CD63 and lysosome membrane | 2001) |
| | protein (CD62P), PAI-1 and CRP. | |
| Diabetic retinopathy | ↓ blood viscosity, improve | (Ren et al., 2000) |
| patients | microcirculation | |
| Coronary heart disease | Improve insulin resistance, and insulin | (Shi et al., 2002) |
| (CHD) patients | resistance related lipid and fibrinolytic | |
| | abnormality. | |
| AMI patients | ↓ the infarction size, ↓free fatty acids | (Xiao et al., 2004) |
| | (FFA), matrix metalloproteinases-9 | |
| | (MMP-9) and CRP. | |
| CHD patients | Simulate the late phase of ischemic | (Xie et al., 2003) |
| | preconditioning via ↓von Willebrand | |
| | factor (vWF:Ag), ↑nitric oxide (NO) | |
| | end ↓endothelin-1 (ET-1) | |
| Angina patients | Anti-angina, reducing both systolic | (Yang et al., |
| | and diastolic blood pressure and | 1990) |
| | diminishing myocardial oxygen | |
| | consumption, and ↑HDL-cholesterol. | |
| UA patients | ↓ frequency of angina events and | (Zhao et al., |
| | consumed doses of nitrates and | 1998) |
| | improving abnormal resting | |
| | electrocardiogram. | |

Besides its action in vascular system, we believe that puerarin has many unknown actions remain to be explored. We have been in the process of developing puerarin for other clinical uses. We have now discovered that puerarin upregulates PPAR-α in human liver cells and treatment with puerarin has anti-weight gain activities in rats, This is a novel use of puerarn as an anti-obesity agent, Upon extensive search of literature, we believe that we are the first to discover that puerarin upregulates PPAR-α in liver cells and prevents weight gain in animal.

Puerarin is a glycoside of daidzein with unusual carbon-carbon (C-glycoside) at the C-8 position. This C-C linkage makes the glucose unit more resistant to enzymatic hydrolysis and metabolic deactivation than the ordinary carbon-oxygen linkage (O-glycoside, daidzin). This unique linkage differentiates puerarin from daidzin in their in vIvo effects against glucose tolerance. While puerarin improves glucose tolerance, daidzin impairs glucose tolerance in mice (Meezan et al., 2005). Puerarin is rapidly absorbed from the intestine and its metabolites in rats after oral intake and mostly remains unmetabolized in the blood (Yasuda et al. 1995; Prasain et al., 2004; Meezan et al. 2005). This feature differs from daidzin, which metabolizes to daidzein, These results may account for the opposite effects on glucose tolerance (Meezan et al., 2005). Although daidzein was shown to activate PPAR-α (Dang and Lowik, 2004; Ricketts et al., 2005), puerarin has an unique advantage when use clinically for the treatment of obesity in people with diabetes when daidzein may worsen the glucose tolerance (Meezan et al., 2005).

### BRIEF SUMMARY OF THE INVENTION

We discovered that treatment of puerarin in a human liver cell line upregulated peroxisome proliferator-activated receptor (PPAR)-α, a nuclear transcription factor that modulates gene expressions in governing lipid metabolism and plays an important role in obesity. Oral treatment with puerarin in rats has anti-weight gain effects with no apparent toxicity. We are now filing an application for a patent for puerarin and its derivatives to be used as a PPAR-α modulator and can be used for the treatment or prevention of obesity and other diseases related to modulation of PPAR-α in humans and mammals.

There are several other uses of puerarin as therapeutic agents in treatment of cardiovascular diseases and cerebral vascular disease as mentioned in the background. However, puerarin as a PPAR-α modulator is a novel discovery that fulfils the applicable U.S. patent classifications definitions Class 424A as a drug which is capable of preventing, alleviating, treating, or curing abnormal and pathological conditions of the living body by such means as limiting the affect of the disease or abnormality by chemically altering the physiology of the host.

### BRIEF DESCRIPTION OF DRAWINGS

FIG. 1. Chemical Structure of puerarin.

FIG. 2. Expression of PPAR-α in HepG2 cells. Cells were incubated in puerarin (1-100 μM) for 24 hrs. Whole cell lysate were collected and PPAR-α expression was probed using anti-PPAR-α (Santa Cruz, Calif.) in Western blotting. Each lane was loaded with 50 μg protein from cell lysate. Expression of actin was also probed to ensure protein loading.

FIG. 3. Effects of puerarin on the body weights of rats. The rats were fed on a normal chow diet for 5 months starting when they were 4 weeks old. The rats were treated by gavage either with vehicle (normal saline) or puerarin (250 mg/Kg/day in a single dose in normal saline). The data were compared statistically by using two-way ANOVA with n=8 in each group. The treatment group is significantly different from the control group (*** P<0.001) over the 5-month period.

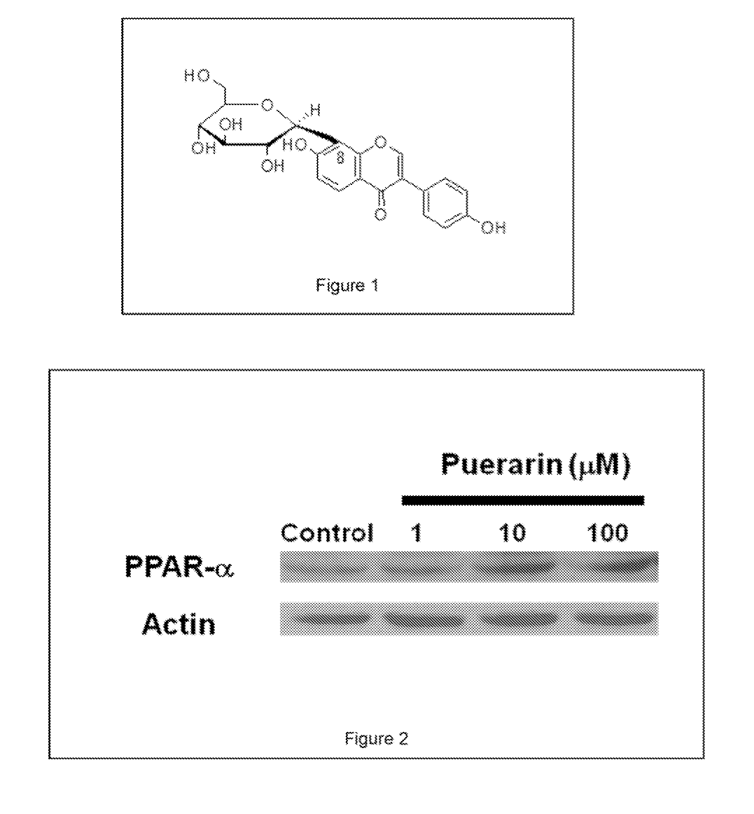

### DETAILED DESCRIPTION OF THE INVENTION

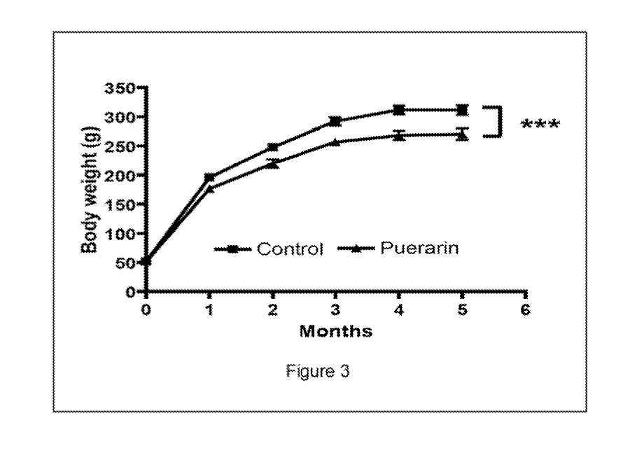

We discovered that treatment of puerarin (1-100 μM) in human liver cell line (HepG2) upregulated the peroxisome proliferator-activated receptor (PPAR)-α (FIG. 2), a nuclear transcription factor that modulates gene expressions in governing lipid metabolism and plays an important role in obesity. In exploring the possible clinical uses of puerarinn we conducted experiments in rats and found that rats that were given puerarin (250 mg/Kg/day) orally were weight 13% less than the control rats in average over 5 months period with no apparent toxicity (FIG. 3). Upon extensive search of literature, we believe that we are the first to discover that puerarin upregulates PPAR-α in liver cells and prevents weight gain in animal. Experimental Procedures, Materials Puerarin was obtained from Natural Pharmacia International, Inc. (Belmont, Mass.). Polyclonal anti-PPAR-α antibody was purchased from Santa Cruz Biotechnology, Inc. (Santa Cruz, Calif.). Fetal bovine serum was purchased from Hydcone Laboratories, Inc. (Logan, Utah). Dulbecco's modified Eagle's medium (DMEM) was purchased from Mediatech, Inc. (Herdon, Va.), Penicillin/streptomycin was purchased from Invitrogen Corp. (Carlsbad, Calif.).

Cell Cultures HepG2 cells was purchased from American Type Culture Collection (Manassas, Va.). They were maintained in DMEM containing 10% heat-inactivated fetal bovine serum and 1% penicillin/streptomycin in tissue culture dishes incubated in 95% air/5% CO₂, at 37° C. The media were changed 2 times per week until the cells were confluent.

Protein immuno-blotting of PPAR-α. HepG2 cells were collected in RIPA buffer (150 mM NaCl, 1% Triton X-100, 1% sodium deoxycholate, 0.1% sodium dodecyl sulfate, 50 mM Tris-HCl, 2 mM EDTA, pH 7.5) with protease inhibitors (Mini-Complete protease inhibitor cocktail tablets (Roche Diagnostics, Mannheim, Germany)] after incubated in puerarin (1-100 μM) for 24 hours. The protein content of the cell lysate was quantified by using a protein assay kit (Bio-Rad Lab,, Hercules Calif.). Protein (50 μg) from the whole cell lysate was loaded in each lane of a polyacrylamide (10%) gel, eletrophoresed and blotted to a polyvinylidene difluoride (PVDF) membrane. The membrane was first probed with a rabbit polyclonal antiserum against PPAR-α, and then with horseradish peroxidase-conjugated donkey anti-rabbit immunoglobulin G secondary antibody. The antibody conjugates were detected by using enhanced chemiluminesence.

### Animal Studies

Spontaneously hypertensive stroke-prone (SHRSP) male rats were fed on a normal chow diet for 5 months starting when they were 4 weeks old. The rats were treated by gavage either with vehicle (normal saline) or puerarin (250 mg/Kg/day in a single dose in normal saline). Their blood pressures and body weights were monitor everyday for 5 months.

### REFERENCES

AGA (2002) American Gastroenterological Association medical position statement on Obesity. Gastroenterology 123:879-881.
Bordet R, Gele P, Duriez P. Fruchart J C (2006) PPARs: a new target for neuroprotection. J Neurol Neurosurg Psychiatry 77;285-287.
Costet P, Legendre C, More J, Edgar A, Galtier P, Pineau T (1998) Peroxisome proliferator-activated receptor alpha-isoform deficiency leads to progressive dyslipidemia with sexually dimorphic obesity and steatosis. J Biol Chem 273:29577-29585.
Dang Z, Lowil: C W (2004) The balance between concurrent activation of ERs and PPARs determines daidzein-induced osteogenesis and adipogenesis. J Bone Miner Res 19:853-861.
Delerive P, Gervois P, Fruchart J C, Staels B (2000) Induction of IkappaBalpha expression as a mechanism contributing to the anti-inflammatory activities of peroxisome proliferator-activated receptor-alpha activators, J Biol Chem 275:36703-36707.
Deplanque D, Gele P, Petrault O, Six I, Furman C, Bouly M, Nion S, Dupuis B, Leys D, Fruchart J C, Cecchelli R, Staels B, Duriez P, Bordet R (2003) Peroxisome proliferator-activated receptor-alpha activation as a mechanism of preventive neuroprotection induced by chronic fenofibrate treatment. J Neurosci 23:6264-6271.
Devchand P R, Keller H, Peters J M, Vazquez M, Gonzalez F J, Wahli W (1996) The PPARalpha-leukotriene B4 pathway to inflammation control. Nature 384:39-43.
Duan S, Li Y F, Luo X L (2000) [Effect of puerarin on heart function and serum oxidized-LDL in the patients with chronic cardiac failure). Hunan Yi Ke Da Xue Xue Bao 25:176-178.
Dwyer J T, Allison D B, Coates P M (2005) Dietary supplements in weight reduction, J Am Diet Assoc 105:S80-86.
Ferre P (2004) The biology of peroxisome proliferator-activated receptors: relationship with lipid metabolism and insulin sensitivity. Diabetes 53 Suppl 1:S43-50.
Guerre-Millo M, Gervois P, Raspe E, Madsen L, Poulain P, Derudas B, Herbert J M Winegar D A, Willson T M, Fruchart J C, Berge R K, Staels B (2000) Peroxisome proliferator-activated receptor alpha activators improve insulin sensitivity and reduce adiposity. J Biol Chem 275:16638-16642.
Hedley A A, Ogden C L, Johnson C L, Carroll M D, Curtin L R, Flegal K M (2004) Prevalence of overweight and obesity among US children. adolescents, and adults, 1999-2002. Jama 291:2847-2850.
Issemann I, Green S (1990) Activation of a member of the steroid hormone receptor superfamily by peroxisome proliferators. Nature 347:645-650.
Jeong S. Kim M, Han M, Lee H, Ahn J, Song Y H, Shin C, Nam K H, Kim T W, Oh G T, Yoon M (2004) Fenofibrate prevents obesity and hypertriglyceridemia in low-density lipoprotein receptor-null mice. Metabolism 53:607-613.
Ji H, Outterbridge L V, Friedman M I (2005) Phenotype-based treatment of dietary obesity: differential effects of fenofibrate in obesity-prone and obesity-resistant rats, Metabolism 54:421-429.
Kersten S (2002) Peroxisome proliferator activated receptors and obesity, Eur J Pharmacol 440:223-234.
Keung W M, Vallee B L (1998) Kudzu root: an ancient Chinese source of modern antidipsotropic agents. Phytochemistry 47:499-506.
Li S (1596) Grand Materia Medica (Ben Cao Gang Mu). Li S M, Liu B, Chen H F (1997) [Effect of puerarin on plasma endothelin, renin activity and angiotensin II in patients with acute myocardial infarction]. Zhongguo Zhong Xi Yi Jie He Za Zhi 17:339-341.
Luo ZR, Zheng B (2001) [Effect of Puerarin on platelet activating factors CD63 and CD62P, plasminogen activator inhibitor and C-reactive protein in patients with unstable angia pectoris]. Zhongguo Zhong Xi Yi Jie He Za Zhi 21:31-33.
Mancini F P, Lanni A, Sabatino L, Moreno M, Giannino A, Contaldo F, Colantuoni V, Goglia F (2001) Fenofibrate prevents and reduces body weight gain and adiposity in diet-induced obese rats. FEBS Lett 491:154-158.
Mandard S, Muller M, Kersten S (2004) Peroxisome proliferator-activated receptor alpha target genes. Cell Mol Life Sci 61:393-416.
Meezan F, Meezan F M, Jones K, Moore R, Barnes S, Prasain J K (2005) Contrasting effects of puerarin and daidzin on glucose homeostasis in mice. J Agric Food Chem 53:8760-8767.
Morris A M, Calsbeek D J, Eckel R H (2004) Lipid metabolism and nutrient partitioning strategies. Curr Drug Targets CNS Neurol Disord 3:411-430,
NTPPTB (1996) Long-term pharmacotherapy in the management of obesity. National Task Force on the Prevention and Treatment of Obesity. Jama 276:1907-1915.
Prasain J K, Jones K, Brissie N, Moore R, Wyss J M, Barnes S (2004) Identification of puerarin and its metabolites in rats by liquid chromatography-tandem mass spectrometry. J Agric Food Chem 52:3708-3712.
Qicheng F (1980) Some current study and research approaches relating to the use of plants in the traditional Chinese medicine. J Ethnopharmacol 2:57-63.
Ren P, Hu H, Zhang R (2000) [Observation on efficacy of puerarin in treating diabetic retinopathy]. Zhongguo Zhong Xi Yi Jie He Za Zhi 20:574-576.
Ricketts M L, Moore D D, Banz W J, Mezei C, Shay N F (2005) Molecular mechanisms of action of the soy isoflavones includes activation of promiscuous nuclear receptors. A review. J Nutr Biochem 16:321-330.
Shi W G, Qu L, Wang J W (2002) [Study on interventing effect of puerarin on insulin resistance In patients with coronary heart disease]. Zhongguo Zhong Xi Yi Jie He Za Zhi 22:21-24.
van Raalte D H, Li M, Pritchard P H, Wasan K M (2004) Peroxisome proliferator-activated receptor (PPAR)-alpha: a pharmacological target with a promising future. Pharm Res 21:1531-1538.
Wolf A M, Colditz G A (1998) Current estimates of the economic cost of obesity in the United States. Obes Res 6:97-106.
Xiao L Z, Huang Z, Ma S C, Zen Z, Luo B, Lin X, Xu X (2004) [Study on the effect and mechanism of puerarin on the size of infarction in patients with acute myocardial infarction]. Zhongguo Zhong Xi Yi Jie He Za Zhi 24:790-792.
Xie R Q, Du J, Hao Y M (2003) [Myocardial protection and mechanism of Puerarin Injection on patients of coronary heart disease with ischemia/reperfusion]. Zhongguo Zhong Xi Yi Jie He Za Zhi 23:895-897.
Yang G, Zhang L, Fan L (1990) [Anti-angina effect of puerarin and its effect on plasma thromboxane A2 and prostacyclin]. Zhong Xi Yi Jie He Za Zhi 10:82-84, 68.
Yasuda T, Kano Y, Salto K, Ohsawa K (1995) Urinary and biliary metabolites of puerarn in rats. Biol Pharm Bull 18:300-303.
Yoon M, Jeong S, Lee H, Han M, Kang J H, Kim E Y, Kim M, Oh G T (2003) Fenofbrate improves lipid metabolism and obesity in ovariectomized LDL receptor-null mice. Biochem Biophys Res Commun 302:29-34.
Yoon M, deong S, Nicol C J, Lee H, Han M, Kim J J, Seo Y J, Ryu C, Oh C T (2002) Fenofibrate regulates obesity and lipid metabolism with sexual dimorphism. Exp Mol Med 34:481-488.
Zambon A, Gervois P, Pauletto P, Fruchart J C, Staels B (2006) Modulation of hepatic inflammatory risk markers of cardiovascular diseases by PPAR-alpha activators: clinical and experimental evidence. Arterioscler Thromb Vasc Bio 26:977-986.
Zhao Z, Yang X, Zhang Y (1998) [Clinical study of puerarin in treatment of patients wth unstable angina]. Zhongguo Zhong Xi Yi Jie He Za Zhi 18:282-284.

1. A compound having the structure of formula I 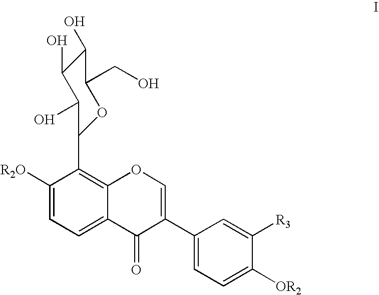
Wherein: R1 is Hydrogen, lower alkyl or hydoxyalkyl, alkylesters; and
R2 is Hydrogen, lower alkyl or hydoxyalkyl, alkylesters, phosphonate; and
R3 is independently selected from hydrogen, lower alkyl, alkoxy, alkyl ester and nitro groups.

2. A method for the treatment or prevention of diseases related to the modulation of PPAR-α having the structure of formula I.

3. A method for treatment or prevention of diseases related to the modulation of PPAR-α including but not limited to obesity in humans or other mammals having the structure of formula I.
